(19) United States
(12) Patent Application Publication
(10) Pub. No.: US 2010/0119458 A1
(43) Pub. Date: May 13, 2010

(54) Compositions Containing Metal Oxide Particles and Their Use

(75) Inventor: Kajsa Uvdal, Linkoping (SE)

Correspondence Address: PORTER WRIGHT MORRIS & ARTHUR, LLP;INTELLECTUAL PROPERTY GROUP, 41 SOUTH HIGH STREET, 28TH FLOOR, COLUMBUS, OH, 43215, US

(73) Assignee: Spago Imaging AB

(21) Appl. No.: 12/525,385

(22) PCT Filed: Jan. 10, 2008
(86) PCT No.: PCT/IB08/50083

§ 371 Date: Jan. 15, 2010

Related U.S. Application Data
(60) Provisional application No. 60/899,995, filed on Feb. 7, 2007.

(51) Int. Cl. A61K 49/06

(52) U.S. Cl. 424/9.32

(57) ABSTRACT

A composition of metal oxide nanoparticles in which individual nanoparticles comprises a core that optionally is coated and the metal oxide comprises a metal oxide lattice in which 5 there are two, three, four or more different kinds of metal ions: A) one of these metal ions is selected among lanthanide ions (typically ions of elements 57-B 71), and) at least one, two, three or more of the other different kinds of metal ions is selected among 10) i transition metal ions of elements of Groups 3b-7b, 8, 1b, 2b other than the lanthanide b ions of elements 57-71, and/or A) lanthanide ions other than the kind of lanthanide ion selected in A. typical metal ion of (A) is Gd3+, of (B.a) is Fe3+ and of (B.b) is Tb3+. 1 5A v method for coating core forms of the particles, and the use of the particles for visualizing various kinds of biological material, e.g. by magnetic resonance, are also provided.

### FIELD OF INVENTION

The present invention relates to compositions of paramagnetic nanoparticles, e.g. super paramagnetic nanoparticles. The nanoparticles have a core comprising metal oxide. The core is in preferred variants coated. Other aspects of the invention comprise a process for manufacturing of the coated forms of the innovative nanoparticles and/or the use of the nanoparticles and their compositions in the study of biological material. The primary use is as contrast agent for imaging of biological material, with preference for magnetic resonance imaging (MRI) possibly in combination with using the same particles as optical probes.

The term “transition metal” will be used in a broad sense in the context of the invention and thus includes elements between group 2b and 3a of the periodic system, i.e. groups 3b, 4b, 5b, 6b, 7b, 8, 1b and 2b with the lanthanides and actinides being part of group 3b.

The term “lanthanides” (Ln) is in the context of the invention used synonymously with the term “rare earth metals” if not otherwise indicated. The term thus includes scandium (Sc), yttrium (Y) in addition to the true lanthanides that are considered to be elements 57-71, i.e. La, Ce, Pr, Nd, Pm, Sm, Eu, Tb, Dy, Ho, Er, Tm, Yb and Lu.

### TECHNICAL BACKGROUND

Magnetic resonance imaging MRI of living material is based on the fact that the spin relaxation times T₁ and T₂ for hydrogen nuclei subjected to an external magnetic field (radiofrequency) vary depending on immediate surroundings, for instance magnetic field, viscosity, temperature etc. A decrease in T₁ leads to an increase in the measured MR signal and a decrease in T₂ to a decrease in the measured MR signal. If a spin echo sequence is used for the measurement, the signal S expressed as a function of scanning parameters can in simplified form be expressed as:

S(TR,TE)=ρe^{-TE/T2}(1−e^{-TR/T1})

where ρ=spin density, TE=echo time and TR=repetition time. Contrast agents that are paramagnetic are used to improve the resolution in the images. They have effects on both T₁ and T₂ but some agents have predominant effects on either T₁ or T₂. Metal ions such as the gadolinium ion (Gd³⁺), e.g. Gd based chelates, and also particles of insoluble salts of certain metals, such as gadolinium oxide (Gd₂O₃) and iron oxide (Fe₂O₃) have been suggested as contrast agents in MRI. Gadolinium (III+) has a predominant effect on T₁ and therefore has been used a positive contrast agent (increased MR signal). Iron (III+) has a predominant effect on T₂ and has therefore been used as a negative contrast agent (decreased MR signal). The relaxation rate (1/Tᵢ, i=1,2 for hydrogen) observed is proportional to the concentration C of a contrast agent used, i.e.

1/Tᵢ(observed)=1/Tᵢ(inherent)+rᵢC

where 1/Tᵢ (observed) is the relaxation rate in the presence of the contrast agent, 1/Tᵢ (inherent) is the inherent tissue relaxation rate, and the proportionality constant rᵢ is called the relaxivity constant for the contrast agent concerned. See Engström et al (Magn Reson Mater Phy 19 (2006) 180-186 and WO 2006031190 (Uvdal et al) and references cited therein.

The effect of a particular contrast agent on the relaxation times of the hydrogen nuclei in a sample and on the MR image depends in a complex way on a number of factors, such as the magnetic moment, the electron relaxation time, the ability to co-ordinate water in the inner or outer co-ordination sphere, rotation of the paramagnetic agent, diffusion and water exchange etc.

MR contrast agents that are used clinically are administered to a patient. This means that the contrast agent has to be given in a form that is not harmful for the patient, remains for a sufficiently long in the patient for the intended use to be performed, is capable of being transported in vivo to the desired location of a body/organ.

To lower toxic effects, gadolinium (III+) has been used clinically as Gd³⁺ in stably chelated form, typically as a diethylene triamine penta acetic acid chelate (DTPA). Nanoparticles containing Gd₂O₃ has so far not been approved for clinical use. The less toxic iron (III+) has been used clinically in the form of Fe₂O₃ nanoparticles. Larger Fe₂O₃ nanoparticles are quickly accumulated in the reticuloendothelial system (RES), have short blood lifetimes and have therefore found use in liver imaging. Smaller Fe₂O₃ nanoparticles have longer blood liftetimes, are escaping RES, and therefore have been considered to have a better potential for general use for imaging in vivo. With respect to clinical use of nanoparticulate forms of Fe₂O₃, the particles have been coated in order to increase their stability against agglomeration and to make them stealthy for the immune system. The coating of particles has typically been degradable in vivo since this would facilitate also degradation of the metal oxide core and thus also the removal of the particles and the metal ions from a patient. However, for contrast agents containing gadolinium (III+) and/or other toxic metal ions a release of metal ions in vivo would be a risk for the patient. It then seems better to design the particles sufficiently small and equipped with a coating that is sufficiently stable to allow for renal elimination of the coated particles. The problem with coatings is that a they might also adversely affect the positive effect a paramagnetic metal oxide core particle has on the image to be recorded. For instance, coatings that are effective in preventing close contact between substances containing hydrogen nuclei (e.g. water) and a metal oxide core would typically mean stable particles but such coatings may at the same time shield the paramagnetic core from interacting with the hydrogen nuclei and lead to poor effects on the relaxation times and on the contrast of the MR image.

### BACKGROUND PUBLICATIONS

1. WO 2005088314 (Perriat et al).

2. WO 2006031190 (Uvdal et al).

3. US 2004/0156784 (Haase).

4. U.S. Pat. No. 4,770,183 (Groman).

5. US 20030180780 (Feng et al).

6. U.S. Pat. No. 6,638,494 (Pilgrimm).

7. CN 1378083 (Sun et al; Chemical Abstracts 8 Mar. 2004, XP002300295 extract from STN data base accession no 2004:184856).

8. Bazzi et al., J. Luminescence, 102-103 (2003) 445-450.

9. Bazzi et al., J. Colloid Interface Sci., 273 (2004) 191-197.

10. Berret et al., J. Amer. Chem. Soc., 128 (2006) 1755-1761.

11. Bridot et al., J. Am. Chem. Soc., 2007, 129, 5076-5085. Publ Mar. 31, 2007).

12. deMellow J. & A., Lab Chip, 4 (2004) 11N-15N.

13. Engström et al., Magn. Reson. Mater. Phy., 19: 180-186 (2006).

14. Feldmann, Adv. Funct. Mater., 13 (2003) 101-107.

15. Feng et al., Anal. Chem. Am. Chem. Soc, Columbus US, 75(19) (2003) 5282-5286.

16. Jun et al., “Magnetic Resonance Nanoparticle Probes for Cancer Imaging,” Nanomaterials for Cancer Diagnosis, Ed. Challa S. S. R. Kumar, Nanotechnologies for the Life Science Vol 7 (147-173) 2007. Wiley-VCH Verlag GmbH & Co. KGaA, Weinheim, Germany.

17. Kawa et al., J. Nanoparticle Res., 5 (81-85) 2002.

18. Louis et al., Chem. Mater., 17 (2005) 1673-1682.

19. Pedersen et al., Surface Science, 592 (2005) 124-140.

20. Söderlind et al., Colloid Interf., 288(1) 140-148 (2005).

21. Tanaka et al., Org. Lett., 9 (2007) 299-302.

All patents, patent applications and publications cited in this specification are incorporated by reference in their entirety.

### SUMMARY OF THE INVENTION

The first aspect of the invention thus relates to a composition that contains nanoparticles that according to the invention are characterized in that individual nanoparticles have a core that comprises a metal oxide lattice in which there are two, three, four or more different metal ions that typically are trivalent:
A) one of these metal ions is selected among lanthanide ions (typically ions of elements 57-71), with preference for those that are paramagnetic and with further preference for those that are capable of exhibiting super paramagnetism when in the form of uncoated core particles within a size interval as defined below, and
B) at least one, two, three or more of the other different metal ions is selected among
   a) transition metal ions of elements of Groups 3b-7b, 8, 1b, 2b other than lanthanide ions of elements 57-71, and
   b) lanthanide ions other than the lanthanide ion selected in A, for instance other than Gd³⁺.

In other aspects, the invention is directed to methods for introducing a covalently attached coating and to methods of visualizing biological material. Other aspects will be apparent in view of the Detailed Description.

### DETAILED DESCRIPTION

The invention is directed to compositions comprising nanoparticles, methods for introducing a covalently attached coating and methods of visualizing biological material.

There is a need for improved paramagnetic nanoparticles, preferably super paramagnetic nanoparticles, that can be used as MRI contrast agents and/or in various fluorescence techniques and neutron capture therapy. See for instance Jun et al. cited herein. This includes improved nanoparticles that will assist in enabling a) increased contrast in the images recorded (spatial resolution), and b) lowering of the detection limit of molecular events and amounts/levels/concentrations of biological compounds and structures, for instance on a cellular level including cell receptors (chemical resolution). Spatial resolutions allowing studies of details down to 1 mm voxel linear dimension (down to 1 mm in size) will thus be facilitated by the use of certain embodiments of the invention with scanning measurements typically being performed during time periods of up to two hours, such as up to 45 minutes. Resolutions down to 10⁻¹ mm voxel linear dimension and even lower, but realistically in most cases above 10⁻² mm voxel linear dimension, are desirable. Improvements (a) and (b) refer to the use of the nanoparticles/compositions as positive contrast agents for the creation of T₁ weighted MR images and/or improved negative contrast agents for the creation of and T₂ weighted MR images. Nanoparticle improvements referring to c) higher tolerance for the immune system of a patient, d) lowered toxicity, e) enhanced stability against release of metal ions (non-degradable, stable coatings) while maintaining efficient magnetic interference between metal ions of the nanoparticles and hydrogen nuclei in the surrounding liquid medium, f) elimination of the nanoparticles from patients by renal filtration without toxic release in vivo of metal ions from the nanoparticles, and/or g) reliable targeting of the nanoparticles inside living material are advantageous.

With respect to MRI, methods for the synthesis of nanoparticulate magnetic MR contrast agents having predetermined and/or controlled properties, e.g. with respect to a) paramagnetic and super paramagnetic properties including effects on the relaxation rates of hydrogen nuclei, b) lifetime of nanoparticles in solution and/or in the circulation in vivo of a body of an animal or an organ thereof, and/or c) toxicity are advantageous.

Various embodiments of the invention provide one or more of these advantages.

The first aspect of the invention thus relates to a composition that contains nanoparticles that according to the invention are characterized in that individual nanoparticles have a core that comprises a metal oxide lattice in which there are two, three, four or more different metal ions that typically are trivalent:
C) one of these metal ions is selected among lanthanide ions (typically ions of elements 57-71), with preference for those that are paramagnetic and with further preference for those that are capable of exhibiting super paramagnetism when in the form of uncoated core particles within a size interval as defined below, and
D) at least one, two, three or more of the other different metal ions is selected among
   a) transition metal ions of elements of Groups 3b-7b, 8, 1b, 2b other than lanthanide ions of elements 57-71, and
   b) lanthanide ions other than the lanthanide ion selected in A, for instance other than Gd³⁺.

The metal ion of (A) typically constitutes ≧10% (atomic %) of the total metal ion content of the core/cores of a single nanoparticle/of the nanoparticles that contains metal ions according to (A) and (B) above, e.g. ≧20% or ≧40% or ≧60% or ≧80%, or ≧90%. The metal ion(s) of (B) typically constitute(s) an essential portion of the content of metal ions other than the metal ion selected in (A), e.g. all of these other kinds of metal ions, or ≦90% (atomic %), or ≦80%, ≦60%, ≦40%, or ≦20% or ≦10%, of the total metal ion content of the core/cores of a single nanoparticle/of the nanoparticles.

For groups (A) and (B.b), the preference is for the true lanthanides, i.e. elements 57-71, and/or for those lanthanides that in oxide form are capable of exhibiting one or more unpaired electrons and/or are paramagnetic either with the lanthanide as the sole metal ion or in combination with another kind of metal ion, such as another transition metal ion.

The transition metals of group (B) that in ion form are typical for the invention are found among elements of Group 3b Sc,Y, La; Group 4b Ti, Zr, Hf; Group 5b V, Nb, Ta; Group 6b Cr, Mo, W; Group 7b Mn, Te, Re; Group 8 Fe, Ru, Os, Co, Rh, Ir, Ni, Pd, Pt; Group 1b Cu, Ag, Au; Group 2b Zn, Cd, Hg; and the actinides (Ac, elements 89-103).

The transition metal preferably should be capable of exhibiting paramagnetism and/or ferromagnetism when in oxide form with particular emphasis of those that are capable of exhibiting super paramagnetism in nanoparticle form, e.g. as an oxide.

Super paramagnetism of a metal ion that is capable of exhibiting this property typically requires that the metal ion is in nanoparticle form, e.g. as an oxide. The sizes of the particles is typically within the intervals specified below with particular emphasis of particles having sizes <25 nm, such as <10 nm.

Preferred transition metals to be included according to group (B.a) in the lattice are selected among elements of period 4, such as among Fe, Co, and Ni, with particular emphasis of elements that in oxide form are capable of exhibiting ferromagnetism. This in particular applies to nanoparticles that are intended for MR imaging.

The lanthanide ion of (B.b) are typically present in particles that are fluorescent based on the presence of a lanthanide ion that is capable of fluorescing. Typical lanthanides with this property are elements 60 (Nd), 62 (Sm), 63 (Eu), 65 (Tb), 66 (Dy), 68 (Er), and 70 (Yb). This kind of lanthanides may be combined with a suitable chrompohore that may be organic. The nanoparticles of the invention may also be fluorescent due to the presence of a non-lanthanide fluorophor that may be organic. A fluorescent nanoparticle of the invention may contain either or both of a fluorescing lanthanide ion and an organic fluorophor. Organic chromophors and fluorophors are primarily present in coated particles and then linked to the coating of the individual particles as discussed elsewhere in this specification.

The nanoparticles/cores may have spheroid forms (that includes particles that are spherical), disc-shaped forms (e.g. circular), elongated forms (e.g. rod-like) and various irregular forms. If not otherwise indicated, the term “size” refers to “mean size”. The term also refers to hydrodynamic size (diameter) or sizes measured as the longest distance between two outer surfaces of a particle.

Uncoated core nanoparticles and the cores in coated nanoparticles that contain the metal ions as given above and being present in a composition according to the invention typically have a mean size that is within the range ≦50 nm, such as ≦40 nm or ≦25 nm or ≦20 nm or ≦10 nm or ≦8 nm with preference for ≦6 nm. The lower limits of these intervals are typically 0.5 nm or 1 nm. The coated forms of core nanoparticles are typically larger than the corresponding uncoated cores, for instance with a mean size within the interval ≦250 nm, such as ≦100 nm or ≦50 nm or ≦25 nm or ≦10 nm or ≦6 nm. The lower limit is the same as for the uncoated core forms. The actual size of coated variants will depend on the composition of the coating and the environment in which the nanoparticles are present, for instance propensity of the coating to swell in liquid medium, such as water. In other words coated variants in desiccated form are typically smaller than in wet form, e.g. in a suspension/dispersion (e.g. variants in which the coating is hydrophilic and the liquid milieu is aqueous or otherwise polar). A particular preferred coated variant comprises nanoparticles that in aqueous milieu have sizes within the range of ≦7 nm, such as ≦5 nm in order to promote elimination of the nanoparticles by renal filtration when present in a patient, i.e. excretion of the particles via renal filtration. Though, one should be aware that a polymer coated particle with a larger hydrodynamic radius or diameter than 7 nm also may be filtered out due to deformations (plasticity effects).

The innovative composition may be essentially devoid of individual lanthanide oxide nanoparticles which have sizes outside the mean size ranges and ranges for metal ion composition given above. For sizes of cores in uncoated form this means that the compositions may be essentially devoid of lanthanide oxide cores that have sizes >50 nm, such as >40 nm or >25 nm or >20 nm or >10 nm or >8 nm or >6 nm. For sizes of coated forms this means that the compositions may be devoid of coated lanthanide oxide particles >250 nm, such as >100 nm or >50 nm or >25 nm or >10 nm or >5 nm.

The lanthanide oxide core particles and coated forms thereof may in the innovative composition be monosized (monodisperse), i.e. ≧25%, such as ≧50% or ≧75% or ≧90% of the core particles or the corresponding coated form are within a size interval with the width of ≦20 nm, such as ≦10 nm or ≦5 nm or ≦3 nm or ≦2 nm or ≦1 nm and/or a size distribution with ≧50% or ≧75% or ≧90% of the particles within a size range that is ±75% or ±50% or ±25% or ±10% of the mean particle size. Particles of a composition that are not monosized (monodisperse) are polysized (polydisperse).

A lanthanide oxide core particle of the composition given above is typically homogeneous with respect to metal oxides. In other variants the chemical composition varies in different domains of a core particle, for instance an inner part may have a chemical composition that is different from an outer part, that for instance may have a chemical metal oxide composition as defined above.

Individual cores of the innovative composition should preferably contain one or more single crystalline domains (=crystallites) of the metal oxide composition discussed above. If several domains are present in the same core they preferably should have essentially the same crystal direction. This does not exclude that an innovative composition may contain core particles that comprise amorphous structure together with core particles that comprise crystalline Gd(III)-concentration and otherwise same conditions as illustrated in the experimental part. With respect to the ratio r₂/r₁ for the relaxivity constants r₁ and r₂ ratios higher than that for Gd-DTPA can be accomplished, for instance for the innovative compositions this ratio may be ≧1 under the conditions used in the experimental part. In some instance the ratio may be ≦1. In typical variants and under the same conditions as used in the experimental part the ratio may be ≧1.1, such as ≧1.2 or ≧1.3 with an upper limit that in many variants is 2, such as 1.8 or 1.5 and a lower limit that typically is 0.5 or 1.

The core particle as such can be synthesized according to known principles for metal oxide nanoparticles. See for instance Söderlind et al, J Colloid Interface Sci., 288 (20059 140-148; Feldmann, Adv. Funct. Mater., 13 (2003) 101-107; Bazzi et al, 102 (2003) 445-450; Bazzi et al, J Colloid Interface Sci., 273 (2004) 191-197; Louis et al, Chem. Mater., 17 (2005) 1673-1682; Pedersen et al, Surface Sci., 592 (2005) 124-140; WO 2005 0088314 (Perriat et al); WO 2006031190 (Uvdahl et al); and US 2004 0156784 (Haase et al); U.S. Pat. No. 6,638,494 (Pilgrimm et al).

In principle the synthetic route comprises the following steps: (i) mixing and dissolving a soluble salt, e.g. halide or nitrate, of the desired metal ion and an appropriate hydroxide, e.g. metal hydroxide such as LiOH and NaOH, in the appropriate solvent, (ii) formation of crystal nuclei (nucleation), and (iii) crystal growth. The solvent should be selected such that the desired metal oxide is insoluble compared to the starting salt and hydroxide compound. The various steps are carried out while heating the mixture to a temperature that typically differs between different steps. Step (iii) is typically starting while step (ii) is on-going. Size, size distribution and morphology (e.g. crystalin) of the particles will depend on temperature, concentrations, incubation times, additives etc. See the experimental part and the publications cited.

Promising preliminary results for the manufacture of core particles to be used in the invention have been accomplished by carrying out the three steps in a flow system comprising a first region for step (i), a second region for step (ii) and a third region for step (iii) and transporting the reaction mixture through the regions in the order given during the process. Individual regions may or may not have separate temperature control functions allowing independent heating of a region if necessary. The process can be run in a continuous mode. The use of miniaturised flow systems will facilitate still better control of variables that determine crystal structure or both structures in the same core particle. Thus, in a typical composition according to the invention at least 10%, such as at least 25% or at least 50% or at least 75% of the nanoparticles/cores that have sizes and metal ion composition as discussed above comprise crystalline structure. It can be envisaged that in preferred variants 100% or close to 100% of the nanoparticles/cores of a composition according to the invention should exhibit crystalline structure, i.e. ≧75%, such as ≧80% ≧90%. The crystalline cores typically comprise one or more crystalline structure selected amongst metal oxide structure, perovskite structure, garnet structure etc. Metal oxide structure in particular applies when the lanthanide ion according to (A) is selected to Gd³⁺. Garnet structure and perovskite structure in particular apply to nanoparticles/cores in which the metal ion according to (B.a) has been selected to Fe³⁺, typically with the metal ion of (A) then being selected to Gd³⁺. The term crystalline structure includes crystalline-like structures, for instance crystalline structures that have been somewhat distorted by the replacement of a lanthanide, and also other ordered structures.

The nanoparticles in a composition according to the invention may be porous or non-porous. Non-porosity in particular should apply to the metal oxide core of coated particles. A composition according to the invention may contain nanoparticles in which there are both porous and non-porous cores. Porosity refers to ability for water and/or other liquids to penetrate the core/coat.

Two, three or more nanoparticles may be present in the form of a cluster in which nanoparticles are held together by a matrix that may be in the form of a shell, e.g. a liposome-like structure, or a polymeric network. Each cluster may contain <500 or <250, or <200 or <100 or <50 of the nanoparticles without excluding the possibility that still larger clusters may be formed. The number of nanoparticles in individual clusters within a composition according to the invention may be different or the same.

With the nanoparticles described above for the innovative composition, it is possible to obtain an HMR signal of a magnitude that is at least 50%, such as at least 100%, of the magnitude of the signal obtained for Gd³⁺-DTPA. Even higher HMR signals can be envisaged, such as at least 150%, or at least 200%, or at least 300% or more of the corresponding Gd³⁺-DTPA signal. With respect to relaxation rates (1/T₁ and/or 1/T₂) it is possible to accomplish values that are at least 50%, such as at least 100%, at least 150% or at least 200% of the relaxation rate obtained for Gd³⁺-DTPA. The comparison is made between values obtained for the same growth, and are therefore important for obtaining particles having a desired size, size distribution and morphology (e.g. crystal structure). A miniaturised flow system comprises a microchannel in which the reactions are carried out. Microchannels typically have at least one cross-sectional dimension ≦1 mm.

Important advantages with using a flow system are that a) it can easily be designed to give high productivity, for instance by running the system in continuous mode and/or parallelizing two or more systems/microchannels, and b) it facilitates control of process variables and therefore makes it easier to obtain core particles of a predetermined quality.

Flow systems for preparing nanosized particles, e.g. of metal oxide, have previously been described. See Kawa et al., J. Nanoparticle Res. 5 (81-85) 2002; deMellow J. & A., Lab Chip 4 (2004) 11N-15N (review); Tanaka et al., Org. Lett. 9 (2007) 299-302.

The above-mentioned flow process for the manufacture of core particles for use in coated or uncoated form as contrast agents in the visualization of biological material constitutes a separate inventive aspect as described in our international patent application “Visualization of biological material by the use of coated contrast agents” filed in parallel with this application.

Coatings

In coated variants of the nanoparticles the coating may be hydrophilic or hydrophobic and covalently or adsorptively attached to the core parts of the nanoparticles. A coating is primarily intended to be applied in order to lower leakage of metal ions from the core of the particles to a surrounding liquid.

With respect to stability by means of reduced release of metal ions of (A) and/or (B.a) and (B.b) above in aqueous media, coated particles according to the invention may have the same life-time or a life-time that is at least 150%, such as at least 200% or at least 300% longer, than the life-time for the corresponding uncoated forms (bare forms, core forms). Typically the coated forms of innovative nanoparticles in a composition according to the invention have life-times of at least 1 hour, such as at least 10 hours, or at least 24 hours (1 day), or at least 120 hours (5 days), or at least 168 hours (7 days, a week) or at least 240 hours (10 days) or at least 360 hours (15 days). These comparisons are between results achieved under the same conditions as elaborated in the experimental part with life-time measured as the time it takes for reducing the concentration/amount of the above-mentioned first lanthanide and/or two, three or more other transition metals of the nanoparticles in a suspension to 50% of the starting concentration/amount (half-life time, t_{1/2}). These stability requirements in particular apply to innovative nanoparticles that are to be stored in aqueous liquid media (e.g. water) and/or to be used in vivo in a patient.

Nanoparticles that are to be used as contrast agents in the body of an animal or organ thereof and administered via the blood circulation should be able to remain in the blood circulation for a time sufficient for the desired image to be recorded. The exact desired lifetime will depend on the part of the body/organ to be imaged, the design of the particles (e.g. coating, targeting, size etc), toxicity of the metal ions in the cores etc. As a general guideline suitable lifetimes (t′_{1/2}) of this kind in the circulation are typically found in the interval of at least 5 minutes, such as at least 10 minutes, at least 30 minutes or at least 1 hour or more with upper limits typically being 2 hours, 24 hours or more.

A hydrophilic coating typically exhibits a plurality of polar functional groups containing one or more heteroatoms selected among oxygen, nitrogen, sulphur, such as in ether, thioether, hydroxyl, carbonyl, amido, ester, amino, ester, carboxylic acid etc. Carbonyls typically include keto, carboxylic acids and their derivatives such as salts, esters, amides etc. The ratio between the number of heteroatoms and the number of carbon atoms in a hydrophilic coating is typically ≧0.2 such as ≧0.3 and in a hydrophobic coating ≦0.2, such as ≦0.1. These figures in particular apply to the surface in contact with a liquid when the particles are dispersed in the liquid. The coating may carry positively and/or negatively charged groups. It is suggested that hydrophilic coatings, may have advantages, e.g. exhibiting no charged groups or various types and combinations of anionic, cationic and zwitterionic groups. Pronounced hydrophilic coatings typically have swelling properties in aqueous liquid media.

Hydroxyls may be present in monoalcohols and polyalcohols (two, three or more hydroxyls) such as polyhydroxy polymers that include native or semisynthetic variants such as those with mono- and polysaccharide structure (poly=two, three or more) and other carbohydrate structures for instance sugar alcohols, dextran, cellulose, starch, carbohydrate derivatives etc. Synthetic variants of polyhydroxy polymers are for example poly (hydroxy alkyl acrylates) and corresponding methacrylates, In hydrophilic hydroxyl alkyl acrylates/methacrylates it is preferred that alkyl is lower alkyl, such as C2-C4 alkylene including hydroxy ethyl. A particular interesting variant is polyethylene glycol (poly=two or more) that in free form has one or two hydroxy groups and when present in a coating may be devoid of hydroxy groups.

Amides (possibly N-alkyl substituted) may be present in polypeptides (including proteins), synthetic polyamides for instance polymeric polyamides such as polyvinyl pyrrolidones (PVP), polyacrylamides, polymethacrylamides.

Ethers may be present in alkylene oxy groups (—RO—, where R is an alkylene chain of 1-3, preferably 2, carbon atoms) such as repetitive variants (RO—)ₙ (where n is an integer >2, 3 or more, such as <10000, or <1000, <500, <100) with preference for polyethylene glycol groups.

The coating may be stabilised by covalent inter-molecular cross-links between molecules initially used to form the coat. This kind of coatings typically will be in the form of a three-dimensional covalent network. Intra-molecular cross-links may also be present and stabilize the coat.

Adsorptively attached coatings are typically attached to the surface of a metal oxide core via electrostatic interactions including dipole-dipole interactions and co-ordinative forces such as in the chelating of metal ions, and other interactions or bonds having no or a low covalent nature. This kind of attachment includes also so called physisorption. Thus it is well known to synthesize metal oxide nanoparticles coated with low molecular weight compounds such as diethylene glycol, carboxylic acids such as citric acid and oleic acid etc, and high molecular weight compounds such as dextran and other polymers exhibiting a plurality of the above-mentioned hydrophilic groups, such as polyhydroxypolymers. Low and high molecular weight compounds may be combined in a coat. If an adsorbed compound is covalently cross-linked inter-molecularly after adsorption the molecules in the coating of each nanoparticle will be attached to the core via multi-point attachment with a concomitant stabilisation of the coating to the particle. Compounds used for coating by adsorption may be anionic, cationic, zwitterionic, or contain no charged groups. Low molecular weight compounds typically has molecular weights ≦5000 dalton, such as ≦2500 daltons or ≦1000 daltons.

Covalent attachment of the coating means that the direct bond between the core particle, i.e. the bond linking the coating to the metal ion or to the metal oxide oxygen in the surface of the metal oxide core has a pronounced covalent nature. For binding to metal oxide oxygen this typically means that the oxygen is directly bonded to a carbon atom or to a silicon atom such as deriving from a silane group (—Si—C). For binding to the metal ion this typically means a direct bond between a sulphur atom of a mercapto group (—S—C) and the metal ion. The carbon atoms indicated are typically sp³-hybridised but may alternatively be sp²- or sp-hybridised. The silicon, sulphur and carbon atoms shall be considered parts of the coat.

The coating may comprise an inorganic or organic skeleton that may be of high molecular weight and comprise a three-dimensional network. The attachment of the skeleton to the surface of the core particle may be at one, two, three or more separate positions in the skeleton, thus including various forms of multi-point attachment of the coating.

Typical inorganic skeletons are polysiloxanes in which individual oxygen atoms in the surface of the core particle binds directly to a silicon atom at the same or at different positions in the polysiloxane skeleton. This type of coatings may be accomplished by the use of tetra alkyl ortho silicate (for instance with alkyl being methyl and/or ethyl), possibly in combination with one or more silanes that are capable of introducing organic groups bound via a —Si—C linkage at metal oxide oxygens or reactants introducing organic groups bonded at metal ions of the core particle (see below). In principle any silane containing two or more alkoxy groups, such as methoxy or ethoxy) directly attached to the silicon atom, may result in a polysiloxane skeleton.

Typical organic skeletons are polymeric (including copolymeric) in the sense that they contain repetitive monomeric units that may be the same or different, for instance by being analogous to each other such as in copolymers, polypeptides including proteins, etc. The skeleton may exhibit one or more chains of carbon atoms linked to each other and possibly interrupted at one or more locations by a heteroatom typically selected among nitrogen, oxygen and sulphur. The chains may be branched. In polymeric skeletons the interruptions may be within or between monomeric units.

In certain variants of imaging of tissue material of a patient by MR, PET, fluoresence etc and in neutron capturing therapy there is often a desire to target the innovative particles to a certain part or organ of the material or patient. In analogy with targeting as commonly used in therapy, the coating of the nanoparticles may exhibit a covalently attached targeting group/WO compound (=ligand) that is capable of specifically binding (e.g. by affinity binding) to a target structure specific for the part or the organ to be imaged or treated. The target structure may be specific for a disease such as a diseased organ including specific cancer cells or other types of malignant or disease-related cells. The term “specific” in this context typically means that the target structure is abnormally expressed in a malignancy, on a diseased organ, on malignant cells etc including over-expression, structurally changed, abnormally distributed etc. The targeting group/compound typically exhibits one or more structures selected among (i) peptide structure such as mono and polypeptide structures, (ii) carbohydrate structure such as mono- and polysaccharide structure, (iii) nucleotide structure such as polynucleotide structure, (iv) steroid structure, (v) lipid structure, (vi) vitamin structure, (vii) hormone structure, and (viii) and synthetic mimetics of structures (i)-(vii). Antibodies and antigen/hapten binding fragments of antibodies are commonly used as targeting groups/compounds and exhibit peptide structures. The term “poly” in this context includes oligo, i.e. di, tri, four etc

As already indicated the coating may also exhibit one or more covalently or adsorptively attached organic fluorophors and/or chromophors, e.g. structures that contain carbon-carbon unsaturation and/or aromaticity each of which may be conjugated with one or more double bonded heteroatoms and/or one or more heteroatoms containing a free electron pair. The heteroatoms are selected from oxygen, nitrogen and sulphur.

In principle the nanoparticles of a composition according to the invention may exhibit any group resulting in an ability to analytically detect the particles, e.g. selected among the above-mentioned fluorophors and chromophors. In other words the nanoparticles used in the invention may exhibit an enzymatic group, such as an enzyme, a cofactor, a coenzyme, a substrate, a co-substrate etc, a group containing a radioactive isotope, a member of an affinity pair etc. Suitable affinity pairs in this context are biotin-streptavidin, hapten-antibody (including antigen/hapten binding fragments and constructs) etc.

The nanoparticles in a composition according to the invention are typically A) mixed with a buffer system, e.g. physiologically acceptable, and/or a suitable non-buffering salt, e.g. physiologically acceptable, and/or a carbohydrate, such as mono- or polysaccharide (di, tri etc saccharide), and/or B) in dry powder form or as a dispersion in a liquid, e.g. aqueous liquid such as water. The powder form may have been obtained by lyophilization, air drying, spray-drying etc of a dispersion containing the particles and the proper liquid medium. The powder form of the inventive composition is typically dispersible in the liquid in which the particles are to be used according to the invention. Such liquids are typically physiologically acceptable and/or aqueous (e.g. water). 2-morpholino-ethanesulphonic acid (MES) and 4-(2-hydroxyethyl)piperazine-1-ethane sulfonic acid (HEPES) are examples of potentially useful buffers to be included in the innovative composition or to be used in liquid dispersion media for compositions in powder form. Phosphate buffers may adversely affect the particles. Buffers that enhance aggregation and sedimentation should be avoided. NaCl is a suitable non-buffering salt. Suitable carbohydrates are water-soluble, such as glucose, saccharose, trehalose, etc

In dispersed variants of the innovative compositions the optimal total concentration of metal ion of (A) and (B) is found within a wide range and depends heavily on use and how the use is performed (e.g. stability requirements, diluting steps, in vivo use, administration routes when used in vivo, in vitro experiments (including types of) etc). Optimal concentrations of particles measured as concentration of metal ions of (A), (B) or (A+B) in compositions that are liquid dispersions are thus typically found in the interval 10⁻³-10⁵ μM.

In dispersed variants of the innovative compositions, e.g. with the nanoparticles dispersed in a physiologically acceptable aqueous liquid phase, the optimal total concentration of the metal ion of the metal oxide present in the core particles could reach ≧10 mM with increasing preference for ≧50 mM or ≧100 mM or ≧500 mM or ≧1 M. Upper limits are 4 M or 10 M. Even higher concentrations can be envisaged. The composition to be used in the inventive method typically has a viscosity ≦50 mPas, such as ≦25 mPas or ≦15 mPas, at a concentration of 0.5 M of the metal ion of the nanoparticles, i.e if the composition is a liquid dispersion in which the concentration of the metal ion is above 0.5 M, a viscosity in this range is achievable upon dilution to 0.5 M. For manual bolus injection it is important with a viscosity of no more than 25 mPas, which is the practical limit. To achieve this, it is important that the coating is optimally thin for the particle preparation to be compatible with the demands for high concentration combined with low viscosity. For many contrast agents this limit is reached when the volume fraction of contrast agent particles/molecules in the injectable formulation/composition is around 30%. For a particle preparation with a 5 nm diameter Gd₂O₃ core (containing 1613 Gd ions according to table 1) and a 2 nm coating we get only about 5% volume fraction for a dispersion that is 1 M in metal ion of the nanoparticles. This is very advantageous over classical macromolecular contrast agents where gadolinium chelates are coupled to a macromolecule and the structures are much less compact than the nanoparticles of the current invention.

A further advantage of the inventive contrast agent is that the osmolality can be substantially lower than for particularly Magnevist (GdDTPA) which is as high as 1960 mOsm. With a particulate contrast agent the osmolality will no longer be very dependent on the total number of particles in solution but rather of the fraction of unbound water in the formulation. With the volume fraction of particles below 5% it is likely that some amount of osmotically active small molecules like e.g. lactose, have to be added to the formulation for it to be isoosmotic with blood (285 mOsm) which would be of benefit for the patient.

Other characteristics of dispersed forms of the composition of the invention are that the aqueous liquid phase is a) isoosmotic with the blood of the living organism to which the composition is to be administered, and b) devoid of diethylene glycol (DEG) and residues of unacceptable reactants, by-products and/or solvents from the manufacture of the core particles and/or from the coating process. The term “devoid of” means that the level of such contaminants in the composition is within limits as approved for this kind of composition by a regulatory official, such as FDA in the US or the corresponding authority in Japan or in one or more countries in Europe. For DEG this limit is likely to be below 0.2% of the composition which is the upper limit for DEG in compositions intended for human intake.

Method of Coating of Metal Oxide Nanoparticles

This aspect of the invention is a method that aims at providing coated metal oxide nanoparticles that have a stability and a reduced toxicity that are sufficient for the uses discussed elsewhere in this specification. The coating method can be used for the innovative core particles and also for other metal oxide core particles, for instance of larger sizes and other metal oxide compositions. The methods facilitates obtaining predetermined and controlled properties regarding among others a) stability (primarily measured as release of metal ions), and b) magnetic properties as discussed elsewhere in this specification.

The coating method comprises the steps of:
(i) providing uncoated forms of the nanoparticles (core particles without coat),
(ii) contacting the uncoated forms with a bifunctional reactant that exhibits,
   a) a structure I, that is capable of attaching the reactant to the core particles, and
   b) a structure II that
      b1) is to be a part of the final coat, or
      b2) is transformable to such a part,
      said contacting taking place under conditions allowing attachment of the bifunctional reactant to the core particles; and
(iii) transforming said structure II, if present according to b2, to a part of said coat.

The term “final coat” encompasses all structures of the coat, such as a) targeting structures, b) structures (e.g. fluorophors, enzymatically active groups, isotopes, affinity labels etc), c) structures that only function as coat, d) linker structures that link these structures together and/or to the core of the final particle, e) etc.

Step (iii) comprises that structure II is transformed directly to a structure/structures that is/are part of the final coat, or in one or more steps to intermediate structures that are further transformed in one or more subsequent steps to structures that are part of the final coat.

The attachment of the bifunctional reactant to the surfaces of the core particles may be by adsorptive and/or covalent bonds where a) adsorptive bonds include non-covalent bonds such as electrostatic bonds, dipole-dipole bonds, hydrogen bonds, van-der waals bonds, etc, and bonds created by hydrophobic interactions and by physisorption, and b) covalent bonds includes pure covalent bonds as well as other bonds that have a pronounced covalent character. In other words structure I of the bifunctional reactant is capable of forming adsorptive bonds and/or covalent bonds. Each molecule of the bifunctional reactant may exhibit one, two or more structure I that may be the same or different, i.e. be capable of forming the same or different kinds of bonds to the core. The conditions selected for step (ii) is selected to support formation of the kind of attachment provided by structure I.

In preferred variants structure I is capable of forming at least one or more covalent bonds per molecule reactant between the core particles and the remaining part of the bifunctional reactant.

The various kinds of reactants and conditions utilized are in principle well known in the field. Step (ii) is typically taking place with the core particles dispersed in a liquid medium and with other reactants in dissolved or dispersed form. The liquid medium may be organic or aqueous (such as water). It may be protic or aprotic.

In one preferred variant structure I, comprises a reactive electrophilic group that is capable of forming a covalent bond with metal oxide oxygen on the surface of the uncoated nanoparticles. A typical and useful such reactive group is a C—O—Si group in which a) the carbon atom typically is sp³-hybridised and typically binds directly to one or more additional carbon atoms and/or none, one or more hydrogen atoms, and b) the Si atom binds directly to further groups —O—C and/or to one or more carbon atoms that typically is sp³-hybridised.

In another preferred variant structure I is a reactive nucleophilic group that is capable of forming a covalent bond with a metal ion on the surface of the uncoated particle. A typical such group is a thiol group (SH) in which the sulphur atom is bound directly to a carbon that in turn is directly bound to one or more additional carbon atoms and/or none, one or more hydrogen atoms. The carbon bound directly to the sulphur atom is typically sp³-hybrisised but may possibly be sp²- or sp-hybridised.

In one alternative, structure II typically comprises one or more of the structures discussed for the coating (b1 above). Typically it may be a polyethylene glycol silane (PEG-silane), a ligand-silane, a label silane such as a fluorophore silane, etc.

In another alternative, structure II is transformable to a part of the final coating (b2 above), for instance by exhibiting a reactive centre to which structures of the final coating can be attached, i.e. structures of the kinds defined above for the coat. Typical reactive centres to be used as transformable structures II are: a) nucleophilic centres such as in thiol, amino, carboxy (COOH/COO⁻), hydroxy etc and activated forms thereof, b) electrophilic centres such as in C—O—Si (silanes), silicate ester groups, haloalkyl (e.g. α-halocarbonyl), carbonyl (e.g. esters, halides and anhydrides of carboxylic acid), unsaturation that is α-β to carbonyl etc and activated forms thereof, and c) polymerizable unsaturation, i.e. carbon-carbon double bonds that may be attached to various functional groups such as ether and ester oxygens (vinyl ethers and vinyl esters), carbon atoms of carbonyl groups such as in carboxylic acids and their esters, amides (—CONH₂ that may be N-substituted), etc. Particular examples or reactants of type b2 are (3-aminopropyl)triethoxy silane, tetraethyl or tetramethyl orthosilicate, γ-methacryloxypropyl triethoxy silane and their closest CH₂-homologes and analogues.

The release of metal ions from lanthanide oxide core particles is likely to vary for different parts of the surface of the core, for instance from different parts, such as edges, corners and flat surfaces, of a crystallite if the core particle contains a crystallite. In other words the tendency of release depends on how the metal or the oxygen is exposed to a surrounding liquid. It can therefore be envisaged that there might be advantages of using two different kinds of bifunctional reactants for the formation of a coat: one kind that comprises a structure I that is nucleophilic and reactive with a metal ion in the surface of the core particle and another kind that comprises a structure I that is electrophilic and reactive with oxygen in the surface of the core particle. The two reactions could be allowed to proceed by simultaneous or consecutive incubation of the two bifunctional reactants. In the latter case addition of the electrophilic reactant may precede addition of the nucleophilic reactant or the order may be reversed. In consecutive variants one or more other steps and/or reactions/incubations may be carried out between the two incubations, for instance addition of other bifunctional reactants involved in the formation of coating structures such as targeting groups (ligands), label groups such as fluorophors and/or chromophors, etc. In the simultaneous incubation variant it is important to select reactants and conditions such that direct reaction of the two structures I with each other is not occurring at the expense of the desired reactions with the core surface.

In other variants that utilize different bifunctional reactants the difference is with respect to structure II. Thus a first bifunctional reactant in which structure II is selected from targeting groups (ligands), label groups such as flurophors, chromophors etc, and structures that are transformable to one of these groups, may be combined with a second bifunctional reagent in which structure II is different from structure II in the first bifunctional reactant. Also in these variants the first and the second bifunctional reactant may be used in parallel or subsequent to each other (possibly with intervening additional steps and/or reactants as discussed above).

When two bifunctional reactants are used and incubated in separate steps (consecutively) the lastly incubated reactant is reacted with the nanoparticles as obtained in the closest preceding step.

The structures introduced via steps (ii) and (iii) above may between and/or during steps (ii) and/or (iii) or subsequent to step (iii) be covalently cross-linked (inter- and/or intra-molecularly).

Best Mode for Core Particles and Coatings

The best mode core particles with respect to doping are given in the experimental part. The best mode synthesis is a) carried out in flow systems as given above, and/or b) without contact with air. The best mode coatings at the filing date of this specification comprise using monoalkyl silane reagents as the bifunctional reagent(s) in step (ii) as described in the experimental part to form thin hydrophilic coatings, preferably of monolayer dimensions. Preferences for coatings, doping levels, structures in the coatings, compositions etc are as given in this specification and further elaborated in the above-mentioned international patent application filed in parallel with this application.

Methods of Use

The primary use of the nanoparticles and the compositions of the invention are among others as contrast agents in the study of biological material, for instance for creating images of structures and tissues of living or dead such material (e.g. images based on nuclear magnetic resonance, PET or fluorescence), and/or in therapeutic protocols that comprise irradiation with neutrons (e.g. neutron capture therapy).

The most important imaging technique concerned in the invention is nuclear magnetic resonance imaging (MRI). So far the greatest advantages of particles and compositions according to the invention have been accomplished when designing them for use as as positive contrast agents for the creation of T₁-weighted MR images. This does not exclude that there also may be advantages with designing them to be used as negative contrast agents (for the creation of T₂-weighted MR images). In particular the type of coatings described herein will be used also for other types of contrast agents. Furthermore using our nanoparticles as positive contrast agents for the creation of T₁-weighted MR images does not exclude using them and the compositions of the invention as contrast agents also in other imaging techniques, such as various particle imaging techniques (see below) and/or as negative contrast agents in for instance MRI. Particles and compositions according to the invention may also be used in an innovative manner in fluorescent techniques in which cases fluorescent forms of the particles are used.

The use aspect of the invention also encompasses the use of the nanoparticles/compositions as labels on biospecific affinity reactants for in vitro or in vivo biospecific affinity assays in order to quantitatively or qualitatively characterize various biological entities and structures. This use aspect includes competitive (=inhibition) and non-competitive (such as sandwich) heterogeneous and homogeneous affinity assays and other assays that may employ the innovative nanoparticles as labels of an affinity reactant (=targeting group, ligand) that is used in for instance cell studies, microscopying, histochemistry, determining the presence, absence and/or amount of an analyte, etc and includes qualitative and quantitative measurements.

One subaspect of the use aspect is a method for visualizing biological material, e.g. by magnetic resonance imaging, comprising the steps of: (i) bringing nanoparticles of the composition aspect of the invention into contact with the material, and (ii) recording the image in a per se known manner.

The imaging step (ii) is preferably performed under conditions giving a spatial resolution that as discussed above may be possible with innovative compositions/nanoparticles.

The visualizing may be according to any of the principles outlined above for the use of the nanoparticles. The biological material may be tissue materials, individual cells and other cell samples, organs etc deriving from dead or living material. The material may derive from organisms, such as plants, vertebrates and invertebrates, microorganisms etc. Typical vertebrates are mammals including humans, avians, etc.

The visualizing may be via X-ray imaging, computed tomography (CT), near-IR fluorescence imaging, positron emission spectroscopy (PET), magnetic resonance imaging (MRI), microscopying etc.

Step (i) is carried out according to principles that are well known in the field.

With respect to biological tissue material that is to be visualized when present in an intact animal (including human) or organ, step (i) typically means that the nanoparticles are injected in the form of a dispersion via a blood vessel (intra-arterially or intravenously). For intact animals also other routes may be useful, for instance intramuscularly, orally (with due care taken for protecting the nanoparticles when passing the stomach), intraperitoneally etc. With respect to systemic administration combined with visualizing specific parts or structures of a body or an organ the particles often are equipped with a targeting group. The amount of nanoparticles administered very much depends on what to be visualized, for instance visualizing larger parts of a body or an organ typically requires larger amounts/doses than smaller parts. In the case of searching/visualizing very specific structures or parts, the use of targeted nanoparticles very easily will minimize the amount of nanoparticles needed, for instance when specifically visualizing cancers.

### Experimental Part

Gadolinium Oxide Particles (Undoped)

Surprisingly, it has turned out that it is advantageous for the reliability and reproducibility of the particle synthesis process, to avoid contact of the heated and basic solutions with air. This improves the color of the prepared particle solution from brown-yellow to colorless or, at most, a pale yellow. Also, the reproducibility of the process is enhanced and electron microscopy indicates that the crystals are more regular and show well developed crystal faces. The more well-defined surface of these crystals will make the coating more regular and hence better able to stabilize the crystals. We have also found it to be beneficial to substitute the sodium hydroxide in the process described in Bridot et al., J. Am. Chem. Soc. 2007, 129, 5076-5085, by lithium hydroxide. Unexpectedly, this further increases the fraction of crystals with well developed surfaces. It is believed that these findings will be beneficial also for doped nanoparticles

### Example 1

### Synthesis of DEG Coated Gd₂O₃ Particles using Sodium Hydroxide

Diethylene glycol (DEG, 30 ml) and NaOH (0.3 g, 7.5 mmol), in a round bottom flask, equipped with a magnetic stiffing bar, are stirred under a stream of nitrogen for 30 minutes. The NaOH pellets are first crushed in a mortar and then the required amount is added. The mixture is stirred vigorously and the flask is immersed in a pre-heated oil bath for 30 minutes. The solids are then dissolved. The heating bath is then removed. In a separate flask, also with a nitrogen atmosphere and magnetic stirring, GdCl₃.6H₂O (2.23 g, 6 mmol) is dissolved in DEG (30 ml) by heating to 140° C. under nitrogen for 1 hour. The temperature of the mixture is raised to 180° C. and the sodium hydroxide solution is added in one portion. The solution is vigorously stirred, and kept at 180° C. for 4 hours and then allowed to cool under nitrogen.

### Example 2

### Synthesis of DEG Coated Gd₂O₃ Particles using Lithium Hydroxide

Diethylene glycol (DEG, 30 ml) and LiOH (0.18 g, 7.5 mmol), in a round bottom flask, equipped with a magnetic stiffing bar, is stirred under a stream of nitrogen for 30 minutes. The mixture is stirred vigorously and the flask is immersed in a pre-heated oil bath for 30 minutes. The solids are then dissolved. The heating bath is then removed. In a separate flask, also with a nitrogen atmosphere and magnetic stirring, GdCl₃.6H₂O (2.23 g, 6 mmol) is dissolved in DEG (30 ml) by heating to 140° C. under nitrogen for 1 hour. The temperature of the mixture is raised to 180° C. and the sodium hydroxide solution is added in one portion. The solution is vigorously stiffed, and kept at 180° C. for 4 hours and then allowed to cool under nitrogen.

Gadolinium-Terbium Nanoparticles

Synthesis Procedure:

Terbium-doped gadolinium oxide nanoparticles are synthesized by applying a modified “polyol” method procedure developed by Bazzi et. al. (J. Colloid Interface Sci., 273 (2004) 191-197). For the 5% Tb-doped Gd₂O₃, 5.7 mmol of GdCl₃.6H₂O and 0.3 mmol of TbCl₃.6H₂O are dispersed in 30 mL of diethylene glycol (DEG), strongly stirred and heated in a silicon oil bath at 140-160° C. for 1 hour. Addition of 7.5 mmol of NaOH dissolved in 30 mL DEG follows. After complete dissolution of the compounds, the solution is refluxed at 180° C. for 4 hours under strong stirring, yielding a yellow-green transparent suspension. For the synthesis of 20% Tb-doped Gd₂O₃, the above procedure is also followed (but adding 1.1 mmol of TbCl₃.6H₂O) except for the addition of NaOH solution. To obtain a coated powdered form of the particles, the as-synthesized suspension is first centrifuged-filtered (0.22 μm) for 30 minutes at 40° C. until complete collection of the fluid. This step is done to remove any large size agglomeration of the particles. The filtered suspension is heated to 140-160° C. with stirring, and 1 mmol of NaOH with either 1.5 mmol of citric acid monohydrate (CA) or dinicotinic acid (NA) dissolved in a small amount of DEG is added. The solution is then refluxed at 180° C. for 30 minutes under strong stirring, yielding a whitish-green dispersion/precipitate. After washing and centrifuging in methanol for several times and then drying under vacuum, an off-white powder is collected.

Characterization of Nanoparticles

The rare-earth oxide synthesized Gd₂O₃ doped with terbium element has mostly circular shaped particles with an average size of 3-7 nm in diameter as revealed on high resolution transmission electron microscopy micrographs (TEM). The particles appear as a regular crystalline lattice, showing the (222) planes (d≈3.2 Å), superimposed on an amorphous background. The powders obtained after precipitation with either citric acid (CA) or dinicotinic (NA) acid reveal different morphologies under scanning electron microscopy (SEM). The CA-coated nanoparticles show porous sponge-like structures while the NA-coated nanoparticles appear like agglomerated spherical structures with open cavities.

The Tb-doping level and chemical composition of the nanoparticles are analyzed with X-ray photoelectron spectroscopy (XPS) and energy dispersive X-ray spectroscopy (EDX). The Tb to Gd atom ratios of 5% Tb- and 20% Tb-doped Gd₂O₃ are found to be 0.055±0.004 and 0.226±0.031, respectively. The results further show that Tb exists only as an ion serving as a dopant to the gadolinium oxide particle. Successful coating with DEG, CA and NA is verified by both XPS and IR analysis.

The photoluminescence (PL) spectra of the powder are consistent with earlier findings for similar nanoparticles with four emission peaks between 460 and 640 nm for excitation at 266 nm (Louis et al., Chem. Mater. 17 (2005) 1673-1682).

The nanoparticles can be coated covalently as said elsewhere in this specification, for instance with various bifunctional silanes as described for the iron containing nanoparticles studied in the subsequent patent example.

Gadolinium-Iron Nanoparticles

Synthesis Procedure:

The procedure is essentially as outlined in the publications cited above.

Reference particles (non-doped Gd₂O₃ nanoparticles): 2.71 g of Gd(NO₃)₃ or 2.2 g of GdCl₃ (6 mmol) is dissolved in 30 ml of DEG and heated under reflux and with magnetic stiffing. Then 0.3 g of NaOH (7.5 mmol) in 30 ml of DEG is added, at 95° C. for Gd(NO₃)₃ and at 140° C. for GdCl₃. The reaction is then allowed to proceed at 140° C. for 1 h whereafter the temperature is raised to 180° C. for 4 h.

Fe doped Gd₂O₃ nanoparticles: Gadolinium nitrate Gd(NO₃)₃.6 H₂O (1.9 mmol), Fe(NO₃)₃ (0.1 mmol), NaOH (2.5 mmol) and deionized water (six drops) are added to about 15 ml of diethylene glycol (DEG) (doping level (Fe/(Fe+Gd))=5%). The mixture is stirred and heated to 140° C. When the reactants are dissolved the temperature is further increased to 180° C. and maintained constant for 4 hours. A precipitate is formed which is separated by centrifugation and washed several times with methanol. Gd(NO₃)₃ can be replaced with GdCl₃ which is likely to result in smaller nanoparticles.

By increasing the Fe/(Fe+Gd) ratio in the reaction mixture to 10%, 20% and 50%, the doping level of the obtained nanoparticles is correspondingly increased.

Perovskite Gd₂O₃ nanoparticles (Fe doping level 50%): 1 mmol of GdCl₃.6H₂O and 1 mmol of FeCl₃.6H₂O are added to 10 ml of DEG and heated. When the temperature reaches 180° C., 6 mmol of KOH dissolved in 10 ml of DEG is added. The temperature is further raised to 210° C. and kept at this temperature for 4 h. A dark brown precipitate is formed, separated off by centrifugation and washed twice with methanol. A certain amount of the sample is calcined at 800° C. in air for 3 h. The supernatant from the centrifuging is heated at 500° C. for 4 h, and the brown powder obtained is washed with deionised water.

X-ray diffractograms (XRD) show peaks attributable to the presence of perovskite, garnet and normal Gd₂O₃ crystal structure in varying amounts in particle material obtained from equimolar amounts of GdCl₃ and FeCl₃. The XRD measurements are performed on a Philips APD powder diffractometer, using CuK_{α}radiation (λ=1.5418 Å, 40 kV, 40 mA) and a step-size of 0.025° in 2θ with 4 s/step.

Working up of nanoparticles: Synthesized nanoparticles are centrifuged (Hermle Z513K) using Vivaspin concentrator membrane (polyethersulfone or PES, Vivascience Sartorius, Hannover) for 30 min. Filters with pore size 0.2 μm, 100 000 molecular weight cut off (MWCO) and 50000 molecular weight cut off (MWCO) are used. The speed is set to 1750 rpm and the temperature is set to 40° C. A syringe driven filter with pore size 0.22 μm (Millex® GV Filter Unit 0.22 μm. Durapore® PVDF membrane, Millipore, Corrigtwohill) is also tested. The results are evaluated using dynamic light scattering (DLS).

Dialysis is performed both to remove excess DEG and in later steps unreacted molecules used for functionalization (e.g. silanes). To remove DEG, the suspension is dialyzed against Milli-Q water with a 1000 MWCO membrane (SpectraPor 6, flat width 18 mm, SpectrumLabs, Rancho Dominguez Calif.) on a magnetic stirrer. The water is replaced at least three times the first day and then two times every following day. The ratio of nanoparticle suspension to water is ideally 1:1000. To evaluate the effect of dialysis time on agglomeration, a nanoparticles suspension filtered with Vivaspin 0.2 μm is dialyzed for 48, 72 and 96 h and the result is evaluated using DLS. To remove unreacted species after functionalization steps, both 1000 MWCO and 10 000 MWCO filters are used. Membranes 10 000 MWCO with a flat width of 12 mm and 18 mm are used. The former gives a quicker dialysis but the latter is easier to use and less expensive. Dialyzed suspensions are stored at 4° C.

Size fractionation: The nanoparticles of a batch can be fractionated into size fractions by using Vivaspin 20 ultrafiltration spin columns in a Rotina 35R Centrifuge (Hettich Centrifugen) and filters of decreasing MWCO by filtrating nanoparticles in the filtrate from a filter of higher MWCO through a filter of lower MWCO. The filters of 100000 MWCO, 50000 MWCO, 30000 MWCO and 10000 MWCO which correspond to cut-off sizes 13.3 nm, 6.67 nm, 4 nm, and 1.33 nm when used consecutively in the given order will thus give four defined size fractions, i.e. nanoparticles collected on each filter plus the nanoparticles in the filtrate passing through the 10000 MWCO. The nanoparticles collected on top of the 100000 MWCO filter are discarded since they contain various types of aggregates of undefined sizes and composition.

Measurement of particle sizes: This is carried out by dynamic light scattering (DLS) and transmission electron microscopy (TEM). DLS: The particle size of a colloidal suspension of the above-mentioned perovskite (not heated to 800° C.) material is measured in AV/DLS-5000 system (Lange). The optimal counting rate is about 250 mHz, and normalized intensity correlation function curves are carefully fitted with an exponential algorithm of the second order (200 grid points). The hydrodymic radius for particles of the suspension is found to be 4.8±0.3 and 5.7±1.0 nm. TEM: These studies are carried out with a Philips CM20 ST electron microscope, operated at 200 kV, and a FEI Tecnai G2 electron microscope (200 kV). Samples for TEM analysis are prepared by dissolving in methanol as-synthesized, non-dialyzed products. The dispersion is dried on amorphous carbon-covered copper grids. By the use of TEM images taken at about 500000× magnification size distribution histograms are built from which an average size can be estimated. An average size of 3.5 to 4.0 nm is estimated (crystal core) for the perovskite material.

Functionalization:
a) Silanization of the nanoparticles by the use of a hetero bifunctional silane with a subsequent further functionalization, e.g. PEG-ylation (two-step PEG-ylation procedure). Filtered nanoparticles are sonicated for 15 minutes, in order to break agglomerates. 1 ml of the nanoparticles in a water suspension (typically dialysed in a previous step) is then placed in an eppendorf tube and 50 μl of the bifunctional silane, e.g. 3-aminopropyl triethoxy silane, is added followed by vortexing and 1 h of sonication. During the reaction the silane function binds to the surface of the nanoparticles leaving the other function, e.g. an amino function, free for the subsequent functionalization step, e.g. introduction of hydrophilic polymers such as polyethylene glycol (PEG-ylation). If needed the silane is added together with a solvent with due care taken for favouring reaction between silane and nanoparticles compared to polymerisation of the silane. 10 μL of Milli-Q is then added whereafter the suspension is sonicated for 1 h and placed on a mixer table overnight to give a total reaction time of 20 h. Purification of the silane-coated particles is performed by dialysis against Milli-Q for 48 h with a 1000 MWCO membrane. The same procedure was also performed with 0.5 and 10 μL of the silane. This functionalization is done with 3-aminopropyl triethoxy silane (APTES).
b) Silanization by the use of a bifunctional PEG derivative (one-step PEG-ylation procedure). 3-mercaptopropyl triethoxysilane (MPTES) and a hetero bifunctional Mal-PEG-NHS derivative (Mal=N-maleidyl linked via a spacer (—(CH₂)ₙCO—) to the oxygen in one terminal of PEG and N-succinimidyl linked via a spacer (—CH₂)ₙ.COO—) to the oxygen in the other terminal of PEG (n and n′=an integer>0) are in a prestep reacted with each other under conditions permitting the mercapto group to form a thioether bond with the C—C double bond in Mal. 15 mg of Mal-PEG-NHS (3 μmol) is dissolved in 300 μL of ethanol using sonication since heat is required to achieve dissolution. Then 0.5 μL of MPTES is added and the reaction is allowed to proceed for 1 h in an ultrasonic bath. Next, 1 ml of Gd₂O₃-DEG nanoparticle suspension, filtered and dialyzed for 72 h, is added followed by vortexing and sonication for 2 h. The tube is then placed on a mixer table overnight to give a total incubation time of at least 20 h. To remove excess of Mal-PEG-NHS and MPTES, dialysis is performed against Milli-Q for 48 h using a 10 000 MWCO membrane. The same procedure is done using 5 mg of Mal-PEG-NHS with 0.05 μL MPTES and 10 mg of Mal-PEG-NHS with 0.1 μL MPTES. The NHS group of the thus NHS functionalized nanoparticles can then be further functionalized with targeting groups, labels such as fluorophors and the like, etc exhibiting an amino group.
c) Silanization by the use of PEG silanes, such as PEG-triethoxy silane (one-step PEG-ylation procedure). This kind of silanes (Mw_{PEG}=4000 and 5000 daltons) is reacted as outlined above for other silanes, for instance with the PEG moiety in mono methoxylated form.

Magnetic Properties and Stability of Nanoparticles

Measurement of stability/dissolution of nanoparticles: The desired nanoparticles synthesized as described above and dispersed in MilliQ water are prepared for seven days of dialysis (1000 MWCO dialysis membrane). The concentration/content of Gd(III) in the dispersion as a function of dialysis time are done at three different occasions, i.e. before dialysis, after five days and after seven days. The dialysis is performed at room temperature. The Gd content in the nanoparticle suspension is analyzed by Inductively Coupled Plasma Mass Spectrometry (ICP-MS), Analytica.

15 MRI measurements: See Engström et al., Magn Reson Mater Phy, 19 (2006) 180-186.

Results of Comparative Studies of Different Nanoparticles (Relaxation Rates and Stability):

The results indicate that paramagnetic nanoparticles suitable for magnetic resonance imaging can be synthesized with predetermined and/or improved properties, e.g. with predetermined and/or improved relaxation rates (1/T₁ and 1/T₂), relaxivities (r₁ and r₂) and stability/lifetimes. This is illustrated by the finding that a) PEG silane functionalized Gd₂O₃ nanoparticles have a high 1/T₁ and 1/T₂ (T₁ (1 mM)=0.012 ms⁻¹) and a fast dissolution rate (short lifetime) (t_{1/2}=4 days), b) PEG silane functionalized 5% Fe doped Gd₂O₃ nanoparticles have a high 1/T₁ and 1/T₂ (1/T₁ (1 mM)=0.012 ms⁻¹) and a considerably slower dissolution rate (longer lifetime (t_{1/2}=10 days), and c) DEG coated non-doped Gd₂O₃ nanoparticles have 1/T₁ (1 mM)=0.012 ms⁻¹ and t_{1/2}=14 days. Commercially available and clinically used Gd³⁺-DPTA has under the same conditions lower values for 1/T₁ and 1/T₂ (e.g. 1/T₁=0.005 ms⁻¹). Variations in relaxivities (r₁ and r₂) and in the relaxivity ratio (r₂/r₁) are illustrated by:

| | r₁ | r₂ | |
|---|---|---|---|
| Nanoparticles | mM⁻¹s⁻¹ | mM⁻¹s⁻¹ | r₂/r₁ |
| Gd³⁺-DTPA | 4.1 | 4.7 | 1.1 |
| Gd³⁺ (GDCl₃) | 10.5 | 12.4 | 1.2 |
| Gd₂O₃ PEG-silane dialyzed 120 h | 9.4 | 13.4 | 1.4 |
| GdFeO₃ dialyzed 4 h | 5.1 | 6.1 | 1.2 |
| GdFeO₃ 800° C. dialyzed 4 h | 6.1 | 10.6 | 1.6 |
| GdFeO₃ dialyzed 120 h | 11.9 | 15.2 | 1.3 |
| Gd₂O₃ 5% Fe* dialyzed 16 h | 5.1 | 6.1 | 1.2 |
| Gd₂O₃ 5% Fe* PEG-silane, dialyzed 120 h | 6.1 | 10.0 | 1.6 |

*The synthesis is the same as for GdFeO₃ (perovskite) except that the relative amount of Fe3+ is lowered to 5%.

Measurement of super paramagnetic properties The magnetization behaviour of dried perovskite nanoparticles synthesised as described above is analyzed in a Quantum Design Physical Properties Measurement System (PPMS), by using about 30 mg of nanopowder per scan and a magnetic field range of −30 000 to 30000 Oe. The temperature dependence of magnetization was measured in an applied magnetic field of 100 Oe between 5° and 300° K., using zero-field-cooling (ZFC) and field-cooling (FC) procedures. Results: Super paramagnetism for the studied perovskite nanoparticles cannot be confirmed by the method used. However, the magnetization curve recorded is typical for super paramagnetic nanoparticles with no hysteresis. The saturation magnetization is evaluated to about 7.5 emu/g. The H/T data at 5° K. and 300° K. superposed on a M(H/T) plot point toward super paramagnetic behaviour (Leslie-Pelecky et al., Chem Mater, 8(8) (1996) 1770-1783). A blocking temperature (T_{B}) cannot be determined even if the magnetization values are followed down to a temperature of 2.8 K which points at a still lower blocking temperature for the particles if they are super paramagnetic.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

1. A composition of metal oxide nanoparticles in which individual nanoparticles comprise a core that optionally is coated and the metal oxide comprises a metal oxide lattice in which there are two, three, four or more different kinds of metal ions:
A) one of these metal ions is selected among lanthanide ions (typically ions of elements 57-71), and
B) at least one, two, three or more of the other different kinds of metal ions is selected among
a) transition metal ions of elements of Groups 3b-7b, 8, 1b, 2b other than the lanthanide ions of elements 57-71, and/or
b) lanthanide ions other than the kind of lanthanide ion selected in A.

2. The composition of claim 1, wherein the nanoparticles are paramagnetic, with preference for super paramagnetic.

3. The composition of claim 1, wherein the lanthanide ion according to at least (A) and possibly also (B) is selected among lanthanides that in oxide form are capable of exhibiting one or more unpaired electrons and/or paramagnetism, such as super paramagnetism, either with the lanthanide as the sole metal ion or in combination with another kind of metal ion, such as another transition metal ion of (B) and/or another lanthanide ion.

4. The composition of claim 1, wherein the lanthanide ion of (A) is Gd(III+)

5. The composition of claim 1, wherein at least one of the other two, three or more metal ions is selected among transition metal ions of (B.a) with preference for those that in oxide form are capable of exhibiting paramagnetism, such as super paramagnetism, and/or ferromagnetism either with the selected metal ion or in combination with another kind of metal ion, such as another transition metal ion of (B) and/or a lanthanide ion of elements 57-71.

6. The composition of claim 1, wherein one of the other one, two or more different metal ions of (B.a) is iron (III+).

7. The composition of claim 1, wherein the mean size of the cores of the particles is ≦50 nm with preference for ≦6 nm.

8. The composition of claim 1, wherein the cores are monosized with preferences for the differences in sizes being within 10 nm, such as within 5 nm.

9. The composition of claim 1, wherein the composition is devoid of the individual nanoparticles in which the cores have sizes >50 nm with preference for being devoid of the nanoparticles in which the cores have sizes >6 nm.

10. The composition of claim 1, wherein the nanoparticles are capable of giving an MR signal that is the same or higher than for Gd-DTPA with the comparison being performed in water and with other conditions being the same.

11. The composition of claim 1, wherein the ratio r₂/r₁ between the relaxivity constants r₂ and r₁ is at least the same as for Gd-DTPA with the comparison being performed in water and with other conditions being the same.

12. The composition of claim 1, wherein the core in individual nanoparticles comprises crystalline structure, with preference for essentially all the nanoparticles comprising crystalline structure.

13. The composition of claim 12, wherein the crystalline structure is selected among crystal structure of pure metal oxide, perovskite structure or garnet structure with the metal moiety being selected to permit the selected crystal structure.

14. The composition of claim 1, wherein at least one of the other different metal ions is selected among lanthanides of (B.b) with preference for lanthanides that are capable of fluorescing, such as among elements 60 (Nd), 62 (Sm), 63 (Eu), 65 (Tb), 66 (Dy), 68 (Er), and 70 (Yb).

15. The composition of claim 1, wherein the nanoparticles are capable of fluorescing.

16. The composition of claim 1, wherein individual nanoparticles comprise a coating that is covalently or adsorptively attached to the core of a nanoparticle, said coating possibly being covalently cross-linked.

17. The nanoparticles of claim 16, wherein said coating is hydrophilic.

18. The composition of claim 16, wherein the attachment is covalent, typically (a) via a C—Si linkage in which the Si atom binds to a metal oxide oxygen in the core of a coated nanoparticle, or (b) via the metal part (Me) of the metal oxide (e.g. MeₓO_{y}), e.g. via a Me—S—C linkage.

19. The composition of claim 16, wherein said coating comprises a covalently attached inorganic skeleton that typically is a polysiloxan and said at least one other metal ion is a transition metal according to (B.a), with preference for Fe(III+).

20. The composition of claim 16, wherein the coating exhibits a plurality of one or more hydrophilic groups at least selected amongst hydroxyls, amides, and alkoxy (with preference for repetitive ethylene oxy).

21. The composition of claim 16, wherein the coating exhibits a targeting group or targeting compound (=ligand) that is capable of affinity binding to a bio-organic target structure.

22. The composition according to claim 1, wherein the nanoparticles are A) mixed with a buffer system, e.g. physiologically acceptable, and/or a suitable non-buffering salt, e.g. physiologically acceptable, and/or B) in dry powder form or as a dispersion in a liquid, e.g. aqueous liquid such as water.

23. A method for introducing a covalently attached coating according to claim 16, comprising the steps of:
A) providing uncoated forms of the nanoparticles (core particles without coat),
B) contacting the uncoated forms with a bifunctional reactant that exhibits
a) a structure I that is capable of forming a covalent bond between the core particles and the reactant, and
b) a structure II that
b1) is to be a part of the final coat, or
b2) is transformable to such a part,
said contacting taking place under conditions allowing the formation of the covalent bond; and
C) transforming structure II, if present according to b2, to a part of said coat.

24. The method of claim 23, wherein two different kinds of the bifunctional reactant are used for formation of the coat:
A) a first bifunctional reactant in which structure II comprises a targeting group (ligand), or a group that is transformable to a targeting group (ligand), and
B) a second bifunctional reagent in which the second structure II comprises hydrophilic groups and structures, or a group/structure that is transformable to such coating groups/structures,
which two reactants are incubated consecutively or simultaneously.

25. The method of claim 23, wherein two different kinds of the bifunctional reactant are used for formation of the coat:
A) a first reactant that comprises a structure I that is nucleophilic and reactive with a metal ion in the surface of the core particle, and
B) a second reactant that comprises a structure I that is electrophilic and reactive with metal oxide oxygen in the surface of the core particle,
which two reactants are incubated consecutively or simultaneously.

26. A method of visualizing biological material, e.g. by magnetic resonance imaging, comprising the steps of: (i) bringing nanoparticles of a composition according to claim 1 into contact with the material, and (ii) recording the image in a per se known manner.

27. The method of claim 26, wherein the material is tissue material, for instance from a vertebrate, such as a mammal.

28. The method of claim 27, wherein the tissue material is part of a vertebrate such as a mammal, and step (i) comprises administering a composition comprising a dispersion of the nanoparticles by injection into the vertebrate, for instance into a blood vessel.

29. The method of claim 25, wherein the imaging step (ii) is performed giving a spatial resolution of at least 1 mm, such as at least 0.1 mm, linear voxel dimension.
